(19) United States
(12) Patent Application Publication
(10) Pub. No.: US 2024/0153612 A1
(43) Pub. Date: May 9, 2024

(54) TREATMENT PROCESS OPTIMIZATION

(71) Applicant: uLab Systems, Inc., Memphis, TN (US)

(72) Inventor: Huafeng WEN, Redwood Shores, CA (US)

(73) Assignee: uLab Systems, Inc., Memphis, TN (US)

(21) Appl. No.: 18/462,676

(22) Filed: Sep. 7, 2023

Related U.S. Application Data
(60) Provisional application No. 63/382,326, filed on Nov. 4, 2022.

(51) Int. Cl. G16H 20/40; A61C 7/00; A61C 9/00; G16H 30/40

(52) U.S. Cl. CPC G16H 20/40; A61C 7/002; A61C 9/0046; G16H 30/40

(57) ABSTRACT

Treatment process optimization methods are described herein where one method for optimizing a treatment plan may generally comprise receiving a digital image of a patient dentition, automatically determining one or more movements for the treatment plan based on a state of the patient dentition in the digital image, receiving one or more modifications relating to the treatment plan, and accepting a finalized treatment plan.

### CROSS-REFERENCE TO RELATED APPLICATIONS

This application claims the benefit of U.S. Provisional Patent Application No. 63/382,326 filed Nov. 4, 2022, which is incorporated herein by reference in its entirety.

### FIELD OF THE INVENTION

The present invention relates to methods and apparatus for orthodontics. More particularly, the present invention relates to methods and apparatus for optimizing the treatment planning for orthodontic procedures.

### BACKGROUND OF THE INVENTION

Orthodontics is a specialty of dentistry that is concerned with the study and treatment of malocclusions which can result from tooth irregularities, disproportionate facial skeleton relationships, or both. Orthodontics treats malocclusion through the displacement of teeth via bony remodeling and control and modification of facial growth.

This process has been traditionally accomplished by using static mechanical force to induce bone remodeling, thereby enabling teeth to move. In this approach, braces having an archwire interface with brackets are affixed to each tooth. As the teeth respond to the pressure applied via the archwire by shifting their positions, the wires are again tightened to apply additional pressure. This widely accepted approach to treating malocclusions takes about twenty-four months on average to complete and is used to treat a number of different classifications of clinical malocclusion. Treatment with braces is complicated by the fact that it is uncomfortable and/or painful for patients, and the orthodontic appliances are perceived as unaesthetic, all of which creates considerable resistance to use. Further, the treatment time cannot be shortened by increasing the force, because too high a force results in root resorption, as well as being more painful. The average treatment time of twenty-four months is very long, and further reduces usage. In fact, some estimates provide that less than half of the patients who could benefit from such treatment elect to pursue orthodontics.

Certain major tooth movements can be accomplished with a series of aligners fabricated from sequential tooth movements on the set-up as the treatment progressed. Use of three-dimensional (3D) scanning and use of computers by companies including Align Technologies and as well as OrthoClear, ClearAligner, and ClearCorrect have enabled widespread use of aligners. However, the 3D scanning and computerized planning treatments can be cumbersome and time consuming, particularly for the practitioners in preparing and fine-tuning these treatment plans for fabricating the aligners.

Accordingly, there exists a need for an efficient and cost-effective processes which can be optimized for planning the orthodontic treatment of patients.

### SUMMARY OF THE INVENTION

In the course of planning a treatment for correcting one or more malocclusions in the dentition of a patient, the practitioner may instead directly perform one or more adjustments using a graphical interface (e.g., sliders, as described in further detail herein) on a digital image of a patient's dentition to set the teeth to their desired position, e.g., called VisualRx, such Visual based prescription maybe generated based on former treatments using machine learning, hence in certain cases referred as AI Rx This information can be automatically sent or processed via a cloud-based server and the treatment plan created with any minor modifications made within certain present limits. For example, any modifications may be made where the teeth are limited to a movement of no more than 0.2 mm from the practitioner's desired treatment plan. Based on the treatment, the prescription may be generated and one or more orthodontic appliances such as aligners or bonding template for braces may be fabricated accordingly.

With treatment planning software utilizing aligners or other orthodontic devices, particular treatment planning processes and orthodontic aligners which may be used in any combination with the methods and materials described herein are described in further detail in U.S. Pat. Nos. 10,624,717; 10,335,250; 10,631,953; 10,357,336; 10,357,342; 10,588,723; 10,548,690; 10,881,486; 10,925,698; 10,952,821; 11,051,913; 11,364,098, as well as U.S. Pat. Pubs. 2017/0100208; 2018/0078343; 2018/0078344; 2018/0078335; 2019/0321135; 2019/0343602; 2020/0146775; 2020/0170762; 2020/0205936; 2020/0345459; 2020/0253693; 2021/0106404; 2021/0153981; 2021/0186668; 2021/0282899; 2021/0369417; 2021/0393376; 2022/0054232; 2022/0265395; 2022/0266577. The details of these references are incorporated herein by reference in their entirety and for any purpose.

When a practitioner submits a data file of the scanned digital image of a patient's dentition, the data file may be submitted to a cloud-based server having the software resident. The treatment may be planned accordingly where two or more sets of potential treatment plans may be created automatically which are presented to the practitioner for selection. The options may be reiterated based upon the practitioner's preferences for each selection which may be staged in one or more stages and the preferences of the practitioner may be recorded and automatically processed via artificial intelligence for treatment planning and presentation to the practitioner in additional patient cases. Ultimately, the prescription for a patient may be automatically created based on the preferences which are incorporated into the treatment planning process via the software.

A patient's dentition may be scanned to create a digital data file where the software may automatically create a case and call up the scanner to scan or rescan the dentition as the practitioner may be notified if the patient's dentition requires a rescan. A pre-setup process may initiate where the scanned image file may be associated with particular case file specific to a patient. The software may take the image file and detect any discrepancies such as holes and fill them. The image file may then be segmented and the various features labeled and feature lines may be optionally incorporated as well. The creation of labeling and feature lines may be performed automatically by the software or they may be performed manually by the practitioner or a staff member.

The process may then move to the setup phase where the software may automatically diagnose and present an initial treatment plan to the practitioner, such as an orthodontist, based on their preferences. The preferences may be input for treating a particular patient (or generally for any range of patient dentitions). For example, a practitioner may have a preference for expanding a dental arch for the first several stages to create space so this particular preference may be saved an applied to a specific patient or generally to other cases for the particular practitioner.

The practitioner may also specify any additional treatments for the patient such as extractions, use of attachments, use of interproximal reductions, etc. Alternatively, the practitioner may determine the initial treatment plan and any additional modifications such as any fine-tuning to the treatment plan may be optionally performed subsequently by a third party such as a staff member. Because the initial teeth movements are performed by the practitioner directly, this provides a better understanding of the desired results to any technicians or staff members who may help to modify the treatment plan. Such modifications may be made within specified predetermined limits; for instance, modifications for teeth movements may be limited to a movement of no more than 0.2 mm from the practitioner's desired treatment plan. Once the treatment plan has been optionally modified, the final treatment plan may be further modified by the practitioner or finalized if no further modifications are deemed necessary.

With the treatment plan finalized, the prescription form may be automatically created based on the practitioner's preferences and the post-setup process may be initiated where one or more orthodontic appliances such as aligners may be optionally formed or printed for use by the patient. The prescription may have certain features pre-selected, but the practitioner may change any parameter of the prescription to a particular patient or for individual items as the cases may vary. Any changes made to the prescription may be reflected automatically in the digital model showing the treatment plan. Moreover, the prescription form results in a prescription which may be printed or forwarded to others and a digital model of the teeth showing the results of the treatment plan which may be viewed by others.

An alternative variation of how the treatment planning process may be facilitated by delineating between one example of case types. In this example, a digital stereolithography image file of a patient's dentition may be received via the cloud-based server (or local computing device) from the practitioner and the software may be make an initial determination of whether the case is an easy case or a hard case based on factors determined by the artificial intelligence of the software such as crowding, movements, etc. and then providing a solution for a treatment plan accordingly. In the event that the case is considered an easy case, the software may create the treatment plan for the practitioner who may then determine whether they are satisfied or not satisfied with the treatment plan. If the practitioner is satisfied with the treatment plan presented, the practitioner, staff member, or technician, etc. may make any minor changes or may directly order the one or more aligners to be fabricated. If the practitioner is not satisfied with the treatment plan presented, they may reset the treatment planning in which case the software may re-enter a treatment planning (VisualRx) mode in which the practitioner may again create another treatment plan. Once satisfied, the one or more aligners may be ordered or fabricated.

Depending upon which movements the practitioner selects for developing the treatment plan, the practitioner may develop from the digital model which may present the digital image of the patient's dentition. The practitioner may select from any number of tabs for treating a particular condition such as an overbite, overjet, etc. by selecting the tab, which may further display a detailed focused view and varying display angle of the digital image of the dentition. For example, if the practitioner wishes to change the overjet, the model may jump to the side view and zoom into the incisors, the slider may be used to control the movement and direction of the selected tooth through the graphical interface to correct for the overjet. The digital image may move accordingly and the stage bar may move as well. If another tab such as a midline tab is selected, then the model may turn back to an anterior view, and focus on midline, where the digital image and stage may be changed again to be reflected automatically on the prescription form.

The interface may be used to plan for the movement of various teeth but instead of utilizing graphical sliders, voice recognition algorithms may be programmed into the software to receive auditory commands from the practitioner. For example, the practitioner may state a verbal command such as “overjet” or any other option to bring up the detailed focus view of the model. Additional verbal commands may be presented such as “a little bit more” to verbally control the sliders to adjust the movement of the teeth at some predetermined distance, e.g., 2 mm movements.

The resulting prescription form may include all selections made by the practitioner along with the patient information, stages, and view of the corrected dentition into a single, coherent prescription which may be downloaded, printed, and/or shared by the practitioner.

Yet another variation of treatment planning optimization may be web-based where the software may be resident on a local or cloud-based server or computer. The practitioner, technician, or staff member may initially import the case for a patient as a scanned digital image to the server so that the software may prepare and sort the data, as described herein. The software may make an initial determination as to whether the uploaded case treatment is AI-friendly or not using, for example, the various factors described herein.

In the event that the software makes an initial determination that the case is not AI-friendly, the software may provide the user one or more alternative options. A first option may include directing the practitioner to treatment planning software for a full start-to-finish case completion where the practitioner may create the treatment plan entirely including all movements, stages, etc. without the help of predetermined practitioner preferences used by the software to assist with treatment planning optimization. Once completed, one or more orthodontic appliances may be ordered or fabricated. Alternatively, the software may provide automated assistance in creating the treatment plan, after which the one or more orthodontic appliances may be ordered or fabricated.

In the event that the software makes an initial determination that the case is AI-friendly, the software may automatically prepare the various treatment parameters such as the stages, set-ups, placement of attachments (if applicable), etc. for a fully AI-based treatment plan. This automated treatment plan may be modified automatically within predetermined limits after which the one or more orthodontic appliances may be ordered or fabricated.

Alternatively, the software may take the automated treatment plan and tweak or incorporate minor modifications using the online tools, as described herein, or the minor modifications may be made by the practitioner, staff, technician, etc. and after which the treatment plan may be finalized and accepted. The one or more orthodontic appliances may then be ordered or fabricated.

In yet another alternative, the practitioner, staff, technician, etc. may take the automated treatment plan and simply accept the case and the one or more orthodontic appliances may be ordered or fabricated.

One example of a method for optimizing a treatment plan may generally comprise receiving a digital image of a patient dentition, automatically determining one or more movements for the treatment plan based on a state of the patient dentition in the digital image, receiving one or more modifications relating to the treatment plan, and accepting a finalized treatment plan.

Another example of a method for optimizing a treatment plan may generally comprise receiving a digital image of a patient dentition, automatically determining whether the digital image is suitable for optimization based upon one or more predetermined parameters, determining one or more movements for the treatment plan based on a state of the patient dentition in the digital image, receiving one or more modifications relating to the treatment plan, and accepting a finalized treatment plan.

### BRIEF DESCRIPTION OF THE DRAWINGS

FIG. 1 illustrates a flow diagram showing how a treatment planning process may be generally facilitated.

FIG. 2 illustrates a flow diagram showing how the treatment planning process may be facilitated by delineating between one example of case types.

FIG. 3 illustrates an initial view by a practitioner of a graphical interface prior to case treatment planning.

FIGS. 4A and 4B illustrate examples of various views that a practitioner may use via controls in treatment planning.

FIGS. 5A and 5B illustrate examples of various views that a practitioner may use via voice-activated controls in treatment planning.

FIGS. 6A and 6B illustrate examples of various views that a practitioner may use via various controls in treatment planning.

FIG. 7 illustrates an example of how a practitioner may narrow down treatment planning options over various stages.

FIG. 8A illustrates a resulting prescription prepared through optimization of the treatment planning process.

FIG. 8B illustrates an example of the resulting prescription.

FIG. 9 illustrates a flow diagram showing one variation of treatment planning options.

FIG. 10 illustrates a flow diagram showing another variation of treatment planning.

FIGS. 11A to 11C illustrate examples of various views for each of the different options in treatment planning.

### DETAILED DESCRIPTION OF THE INVENTION

Typically in the course of preparing a treatment plan for a patient, the practitioner, such as an orthodontist, may prepare a textual prescription (Rx) form which is several pages in length for the third party company creating the orthodontic aligners. This third party company will generally receive the Rx form, interpolate the prescription, and create the treatment plan on behalf of the practitioner for their review and approval. If the practitioner wishes to make any modifications to the treatment plan, the communication and modification process may require several iterations between the practitioner and the third party company.

As described herein, in the course of planning a treatment for correcting one or more malocclusions in the dentition of a patient, the practitioner may instead directly perform one or more adjustments using a graphical interface (e.g., sliders, as described in further detail herein) on a digital image of a patient's dentition to set the teeth to their desired position, e.g., called VisualRx. This information can be automatically sent or processed via a cloud-based server and the treatment plan created with any minor modifications made within certain present limits. For example, any modifications may be made where the teeth are limited to a movement of no more than 0.2 mm from the practitioner's desired treatment plan. Based on the treatment, the prescription may be generated and one or more orthodontic appliances such as aligners may be fabricated accordingly.

With treatment planning software utilizing aligners or other orthodontic devices, particular treatment planning processes and orthodontic aligners which may be used in any combination with the methods and materials described herein are described in further detail in U.S. Pat. Nos. 10,624,717; 10,335,250; 10,631,953; 10,357,336; 10,357,342; 10,588,723; 10,548,690; 10,881,486; 10,925,698; 10,952,821; 11,051,913; 11,364,098, as well as U.S. Pat. Pubs. 2017/0100208; 2018/0078343; 2018/0078344; 2018/0078335; 2019/0321135; 2019/0343602; 2020/0146775; 2020/0170762; 2020/0205936; 2020/0345459; 2020/0253693; 2021/0106404; 2021/0153981; 2021/0186668; 2021/0282899; 2021/0369417; 2021/0393376; 2022/0054232; 2022/0265395; 2022/0266577. The details of these references are incorporated herein by reference in their entirety and for any purpose.

When a practitioner submits a data file of the scanned digital image of a patient's dentition, the data file may be submitted to a cloud-based server having the software resident. The treatment may be planned accordingly where two or more sets of potential treatment plans may be created automatically which are presented to the practitioner for selection. The options may be reiterated based upon the practitioner's preferences for each selection which may be staged in one or more stages and the preferences of the practitioner may be recorded and automatically processed via artificial intelligence for treatment planning and presentation to the practitioner in additional patient cases. Ultimately, the prescription for a patient may be automatically created based on the preferences which are incorporated into the treatment planning process via the software.

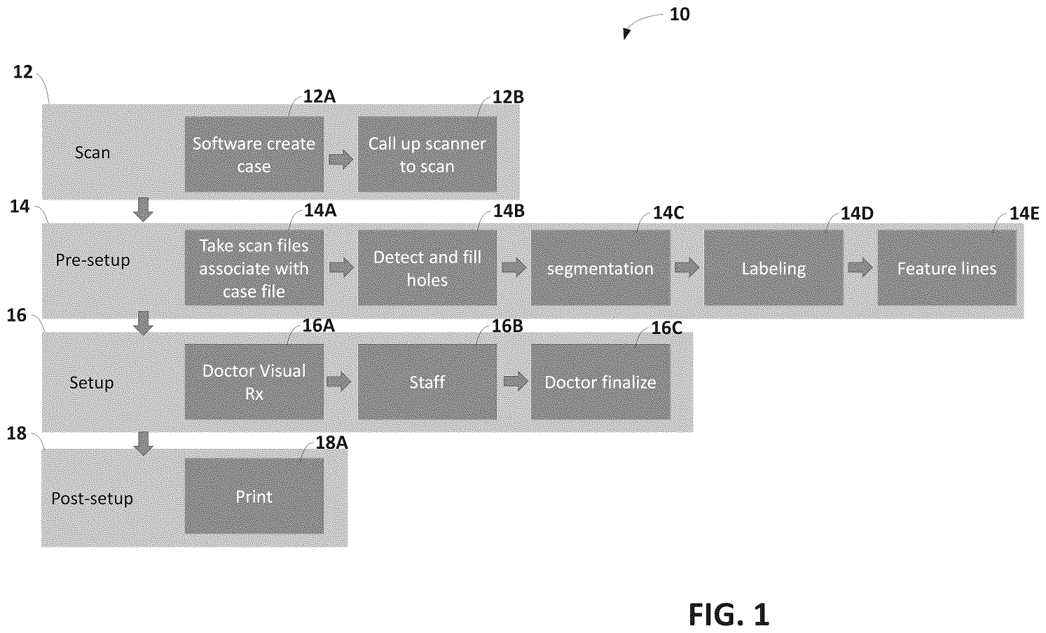

FIG. 1 illustrates a flow diagram 10 of one variation showing one example of a process for treatment planning. A patient's dentition may be scanned 12 to create a digital data file where the software may automatically create a case 12A and call up the scanner to scan or rescan the dentition 12B as the practitioner may be notified if the patient's dentition requires a rescan. A pre-setup 14 process may initiate where the scanned image file may be associated with particular case file 14A specific to a patient. The software may take the image file and detect any discrepancies such as holes and fill them 14B. The image file may then be segmented 14C and the various features labeled 14D and feature lines 14E may be optionally incorporated as well. The creation of labeling 14D and feature lines 14E may be performed automatically by the software or they may be performed manually by the practitioner or a staff member.

The process may then move to the setup 16 phase where the software may automatically diagnose and present an initial treatment plan to the practitioner, such as an orthodontist, based on their preferences. The preferences may be input for treating a particular patient (or generally for any range of patient dentitions). For example, a practitioner may have a preference for expanding a dental arch for the first several stages to create space so this particular preference may be saved an applied to a specific patient or generally to other cases for the particular practitioner.

The practitioner may also specify any additional treatments for the patient such as extractions, use of attachments, use of interproximal reductions, etc. Alternatively, the practitioner may determine the initial treatment plan 16A and any additional modifications such as any fine-tuning to the treatment plan may be optionally performed subsequently by a third party such as a staff member 16B. Because the initial teeth movements are performed by the practitioner directly, this provides a better understanding of the desired results to any technicians or staff members who may help to modify the treatment plan. Such modifications may be made within specified predetermined limits; for instance, modifications for teeth movements may be limited to a movement of no more than 0.2 mm from the practitioner's desired treatment plan. Once the treatment plan has been optionally modified, the final treatment plan may be further modified by the practitioner or finalized 16C if no further modifications are deemed necessary.

With the treatment plan finalized, the prescription form may be automatically created based on the practitioner's preferences and the post-setup 18 process may be initiated where one or more orthodontic appliances such as aligners may be optionally formed or printed 18A for use by the patient. The prescription may have certain features pre-selected, but the practitioner may change any parameter of the prescription to a particular patient or for individual items as the cases may vary. Any changes made to the prescription may be reflected automatically in the digital model showing the treatment plan. Moreover, the prescription form results in a prescription which may be printed or forwarded to others and a digital model of the teeth showing the results of the treatment plan which may be viewed by others.

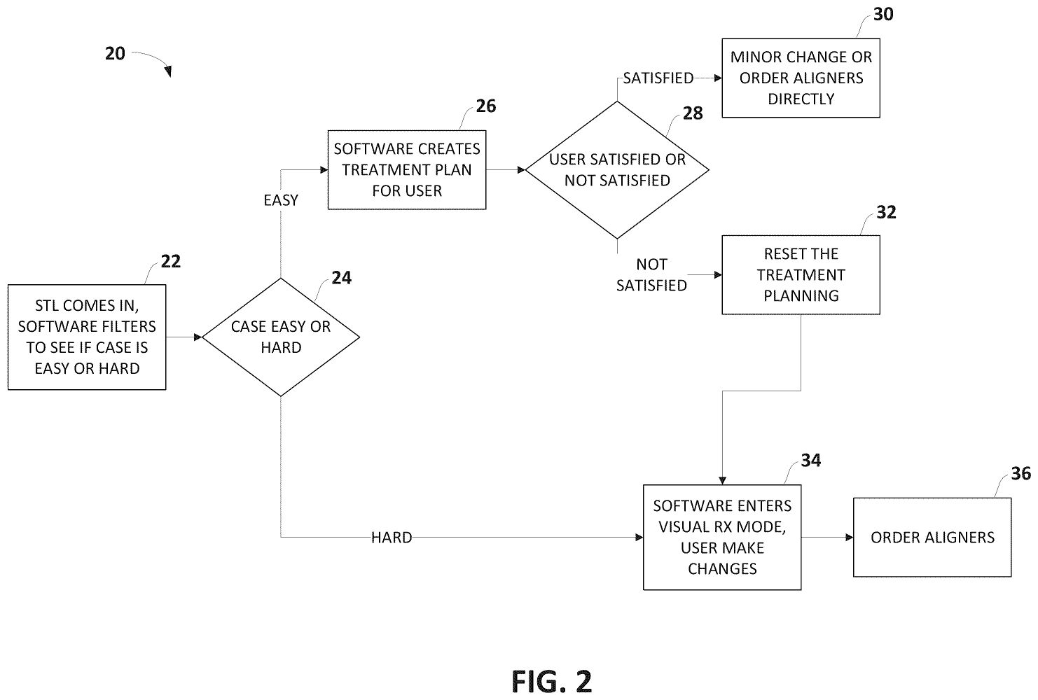

FIG. 2 illustrates a flow diagram 20 showing an alternative variation of how the treatment planning process may be facilitated by delineating between one example of case types. In this example, a digital stereolithography image file of a patient's dentition may be received via the cloud-based server (or local computing device) from the practitioner and the software may be make an initial determination 22 of whether the case is an easy case or a hard case 24 based on factors determined by the artificial intelligence of the software such as crowding, movements, etc. and then providing a solution for a treatment plan accordingly. The following Table 1 provides examples of various parameters with respect to different conditions relating to the patient's teeth and how these parameters may be delineated into mild, moderate, severe conditions. These parameters may be used by the software to help delineate between different levels of difficulty in treatment planning.

TABLE 1
List of parameters and scoring.

| Parameter | | | | |
|---|---|---|---|---|
| (after treatment/ | | | | After |
| set-up) | Mild | Moderate | Severe | treatment |
| Overjet | 0.5-3.5 mm | 3.5-5.0 mm or | >5.0 mm or | |
| | | −1-0.5 mm | <1 mm | |
| Overbite | 1-3.5 mm | 3.5-5.0 mm or | >5.0 mm or | |
| | | 0-1 mm | <0 mm | |
| Midline | | | | |
| (lower to upper) | | | | |
| Alignment | 0 teeth | 1 or 2 teeth | 3 teeth or more | Feature point to |
| | | | | archform <0.5 |
| | | | | mm |
| Post Tooth Contact | 0 teeth | 1 tooth | 2 teeth or more | Space >0.5 mm |
| Anterior-Posterior/ | 0.5 mm | <2 mm | >2 mm | Not fixable |
| Left-Right | | | | |
| Reject Reason | Any severe | 2 moderate | | |
| Acceptance Setting | Essential | Standard | Only give best | |
| | | | result - mostly | |
| | | | standard/full | |

In the event that the case is considered an easy case, the software may create the treatment plan for the practitioner 26 who may then determine whether they are satisfied or not satisfied with the treatment plan 28. If the practitioner is satisfied with the treatment plan presented, the practitioner, staff member, or technician, etc. may make any minor changes or may directly order the one or more aligners to be fabricated 30. If the practitioner is not satisfied with the treatment plan presented, they may reset the treatment planning 32 in which case the software may re-enter a treatment planning (VisualRx) mode in which the practitioner may again create another treatment plan 34. Once satisfied, the one or more aligners may be ordered or fabricated 36.

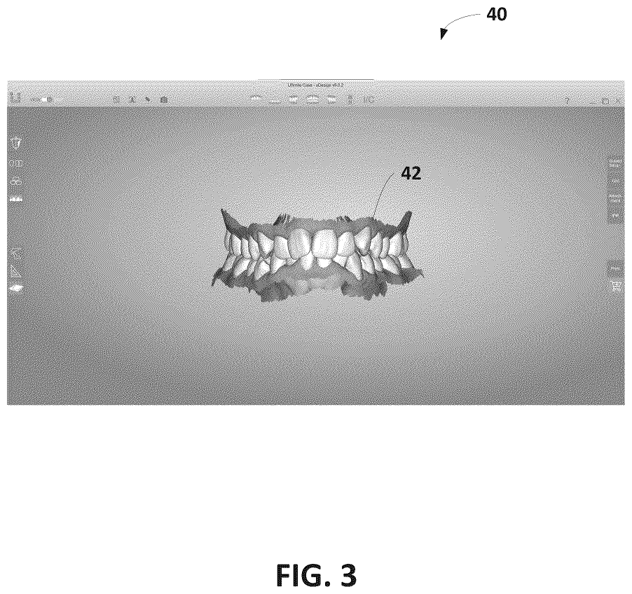

FIG. 3 illustrates an initial view of the digital image of the patient's dentition 42 presented to a practitioner in a graphical interface 40 prior to case treatment planning. The initial view may be presented as a starting point for the setup step 16 once the scan 12 and pre-setup 14 steps have been completed, as described above for FIG. 1.

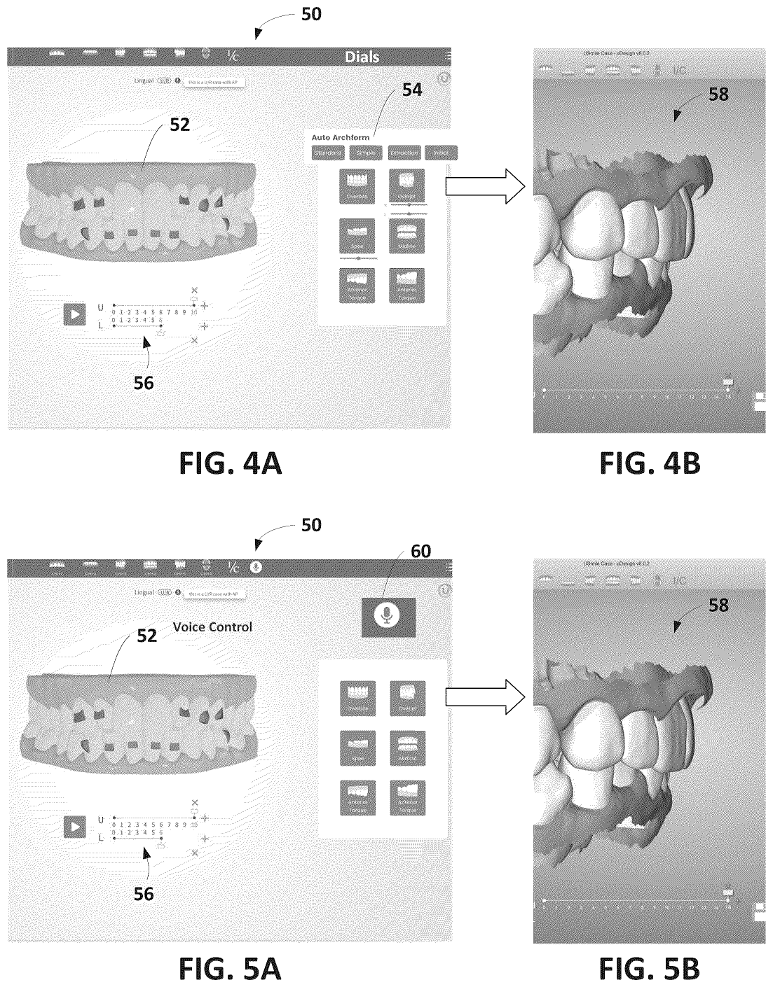

Depending upon which movements the practitioner selects for developing the treatment plan, the practitioner may develop from the digital model, as shown in display 50 of FIG. 4A, which may present the digital image of the patient's dentition 52. The practitioner may select from any number of tabs 54 for treating a particular condition such as an overbite, overjet, etc. by selecting the tab 54, which may further display a detailed focused view and varying display angle of the digital image of the dentition 58, as shown in FIG. 4B. For example, if the practitioner wishes to change the overjet, the model 58 may jump to the side view and zoom into the incisors, as shown, the slider 56 may be used to control the movement and direction of the selected tooth through the graphical interface to correct for the overjet. The digital image 58 may move accordingly and the stage bar may move as well. If another tab such as a midline tab is selected, then the model 58 may turn back to an anterior view, and focus on midline, where the digital image and stage may be changed again to be reflected automatically on the prescription form.

FIGS. 5A and 5B illustrate an alternative user interface that a practitioner may use via voice-activated controls 60 in treatment planning. As described above for FIGS. 4A and 4B, the interface may be used to plan for the movement of various teeth but instead of utilizing graphical sliders, voice recognition algorithms may be programmed into the software to receive auditory commands from the practitioner. For example, the practitioner may state a verbal command such as “overjet” or any other option to bring up the detailed focus view of the model 58. Additional verbal commands may be presented such as “a little bit more” to verbally control the sliders to adjust the movement of the teeth at some predetermined distance, e.g., 2 mm movements.

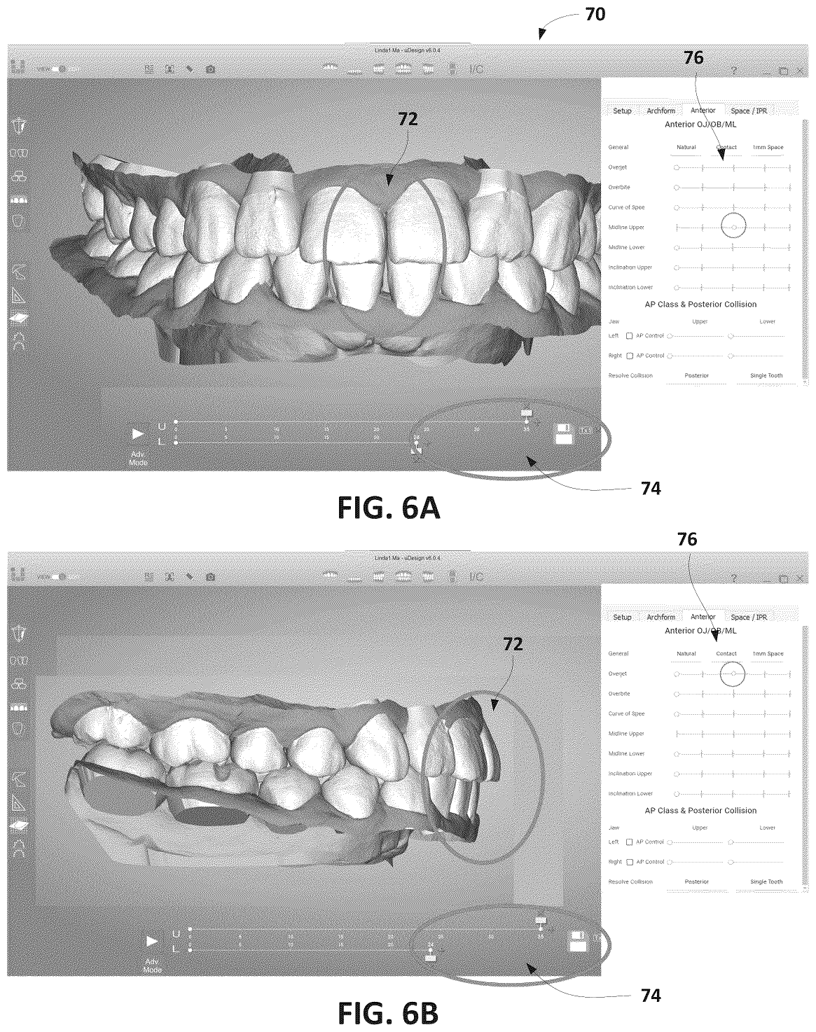

FIG. 6A illustrates another example of the graphical interface for presenting the various views that a practitioner may use via various controls in treatment planning. In this example, the practitioner may select on a feature such as “midline”, which may present the digital image showing the midline view in which the practitioner may adjust one or more of the sliders 76 for which the adjustments are reflected in the image 72 as well as the stage bar 74. FIG. 6B illustrates yet another example where a selection of the tab for “overjet” may automatically focus the model into a profile view where the sliders 76 may be adjusted to reflect the movements in the digital image, stage bar 74, and the prescription form as well.

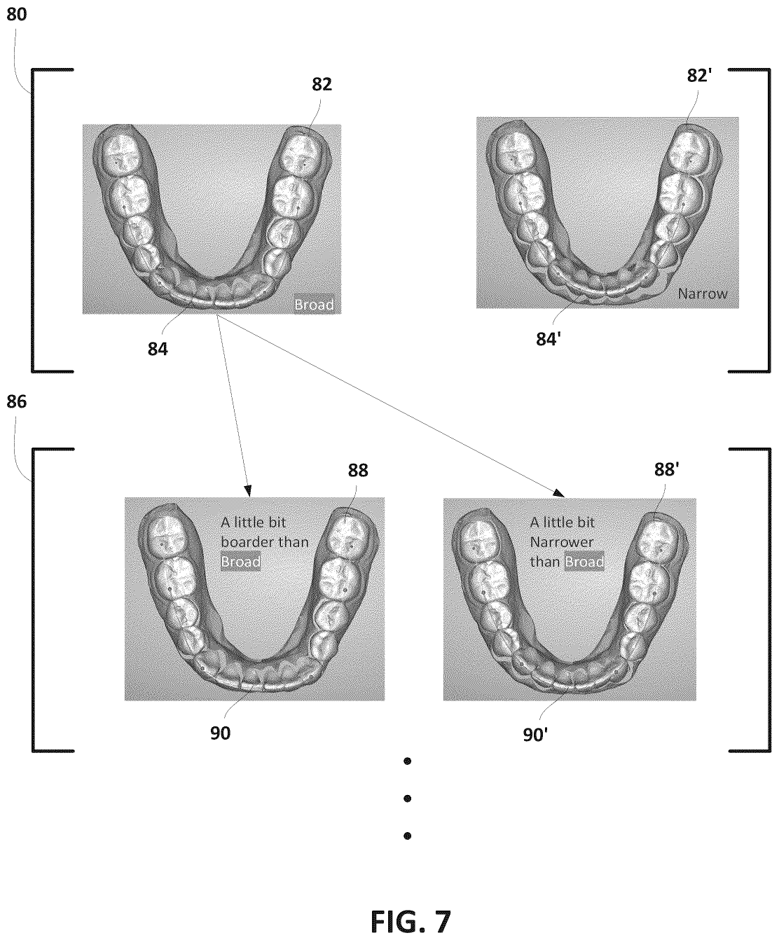

As described above, the automated treatment functionality may be based upon the preferences of the practitioner in treating a particular condition in a patient's dentition. These preferences may be learned by the software algorithm and applied automatically by the algorithm in developing treatment plans for any number of patients. FIG. 7 illustrates an example of how a practitioner may be presented by the algorithm with varying treatment options which are subsequently narrowed in several stages to learn the preferences. For example, in a first stage 80, the practitioner may be presented with two or more choices between differing treatment styles. A first digital model 82 of the dentition may be presented having, e.g., a relatively broad configuration 84 of corrected teeth, and a second digital model 82′ of the dentition may also be presented having, e.g., a relatively narrow configuration 84′ of corrected teeth. The practitioner may select their preferred treatment option in this first stage 80. Based upon the selection, a second stage 86 may be presented between a first digital model 88 having, e.g., a set of teeth which are relatively broader 90 than the initial broad configuration 84, and a second digital model 88′ having, e.g., a set of teeth which are relatively narrower 90′ than the initial broad configuration 84. While selection between two options are shown in each stage, additional options may be provided in alternative variations. This subsequent selection through multiple stages may help to narrow down the practitioner's treatment planning preferences for treatment. It is these preferences for different conditions which may be applied initially by the software to treatment plans for other patients.

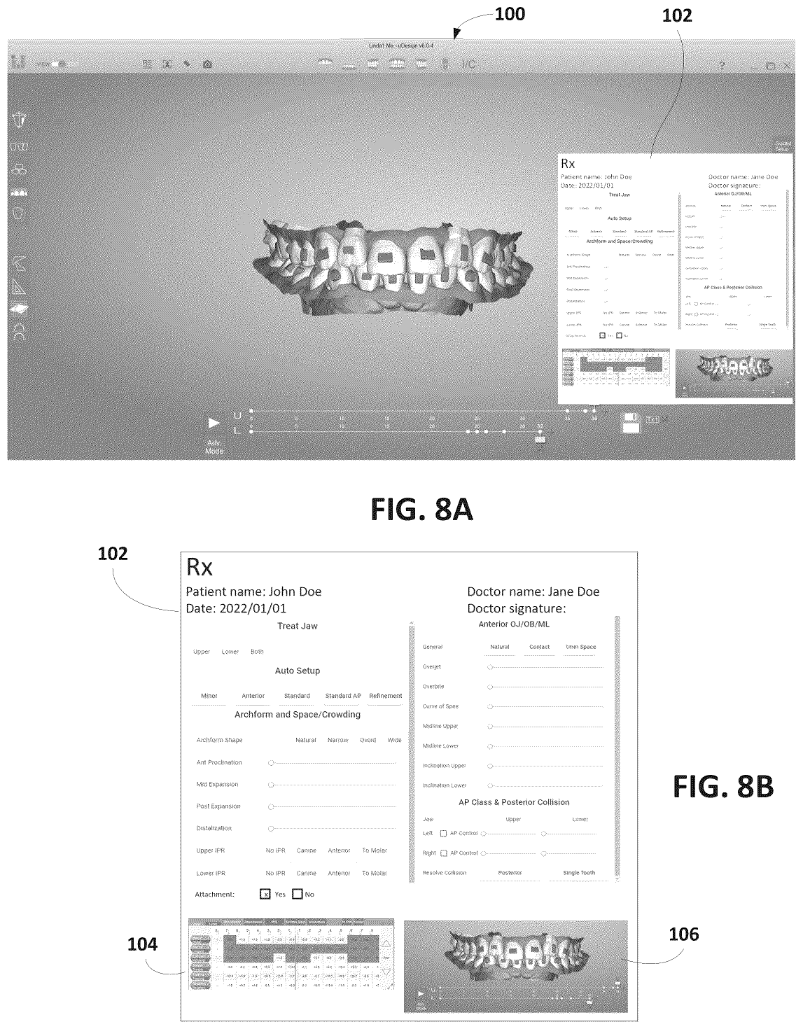

FIG. 8A illustrates an example of the graphical interface 100 illustrating the end result of a treatment following the practitioner's treatment plan. The resulting prescription form 102 prepared by the practitioner through optimization of the treatment planning process is also illustrated with the estimated stages. FIG. 8B shows a detailed example of the resulting prescription form 102 which includes all selections made by the practitioner along with the patient information, stages 104, and view 106 of the corrected dentition into a single, coherent prescription which may be downloaded, printed, and/or shared by the practitioner.

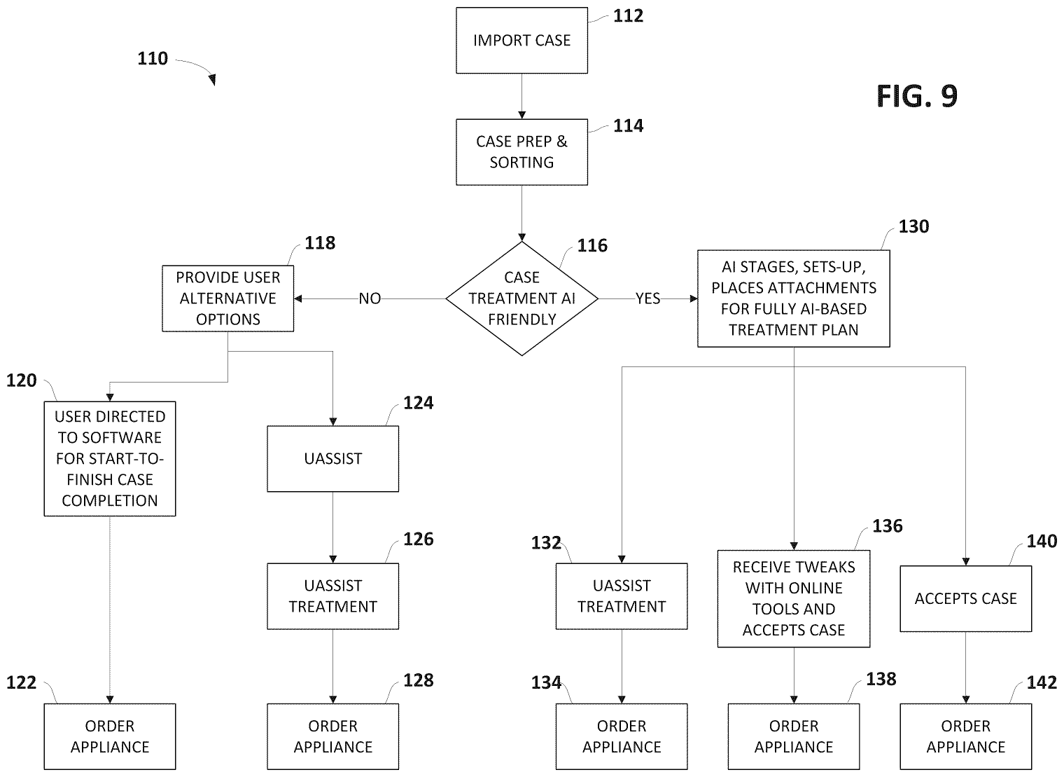

FIG. 9 illustrates a flow diagram 110 showing another variation of treatment planning optimization which may be web-based where the software may be resident on a local or cloud-based server or computer. The practitioner, technician, or staff member may initially import the case for a patient 112 as a scanned digital image to the server so that the software may prepare and sort 114 the data, as described herein. The software may make an initial determination as to whether the uploaded case treatment is AI-friendly or not 116 using, for example, the various factors described herein.

In the event that the software makes an initial determination that the case is not AI-friendly, the software may provide the user one or more alternative options 118. A first option may include directing the practitioner to treatment planning software for a full start-to-finish case completion 120 where the practitioner may create the treatment plan entirely including all movements, stages, etc. without the help of predetermined practitioner preferences used by the software to assist with treatment planning optimization. Once completed, one or more orthodontic appliances may be ordered or fabricated 122. Alternatively, the software may provide automated assistance 124 in creating the treatment plan 126, after which the one or more orthodontic appliances may be ordered or fabricated 128.

In the event that the software makes an initial determination that the case is AI-friendly, the software may automatically prepare the various treatment parameters such as the stages, set-ups, placement of attachments (if applicable), etc. for a fully AI-based treatment plan 130. This automated treatment plan may be modified automatically within predetermined limits 132 after which the one or more orthodontic appliances may be ordered or fabricated 134.

Alternatively, the software may take the automated treatment plan and tweak or incorporate minor modifications using the online tools, as described herein, or the minor modifications may be made by the practitioner, staff, technician, etc. and after which the treatment plan may be finalized and accepted 136. The one or more orthodontic appliances may then be ordered or fabricated 138.

In yet another alternative, the practitioner, staff, technician, etc. may take the automated treatment plan and simply accept the case 140 and the one or more orthodontic appliances may be ordered or fabricated 142.

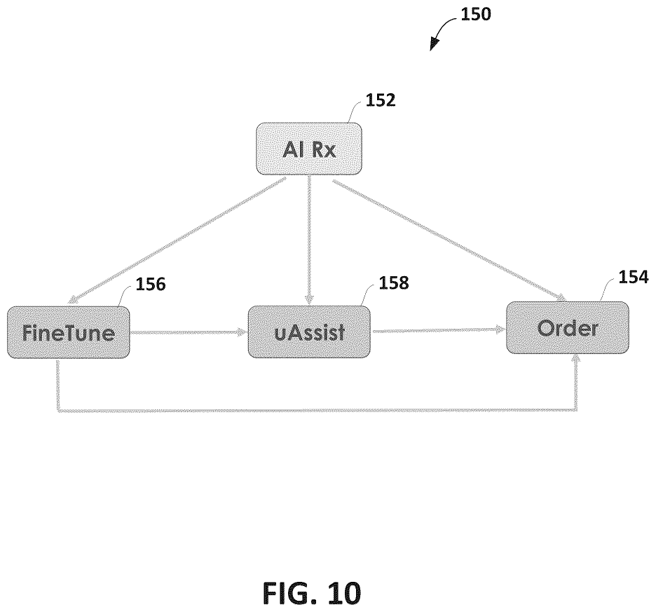

FIG. 10 illustrates another flow diagram 150 showing another variation of treatment planning optimization which may be web-based where the software may be resident on a local or cloud-based server or computer. As described herein, once the case has been imported and prepared, a determination may be made by artificial intelligence through the software of whether the treatment planning may be accomplished by the software 152. If so, the formulated treatment plan may be accepted, prescription prepared, and one or more of the orthodontic aligners may be ordered 154 directly.

Alternatively, if the treatment plan is to be modified of finetuned 156, the treatment may be modified accordingly, as described herein. The modified treatment plan may then be accepted and the one or more orthodontic aligners ordered 154 directly, or the modified treatment plan may be further modified 158 and the one or more orthodontic aligners may then be subsequently ordered 154.

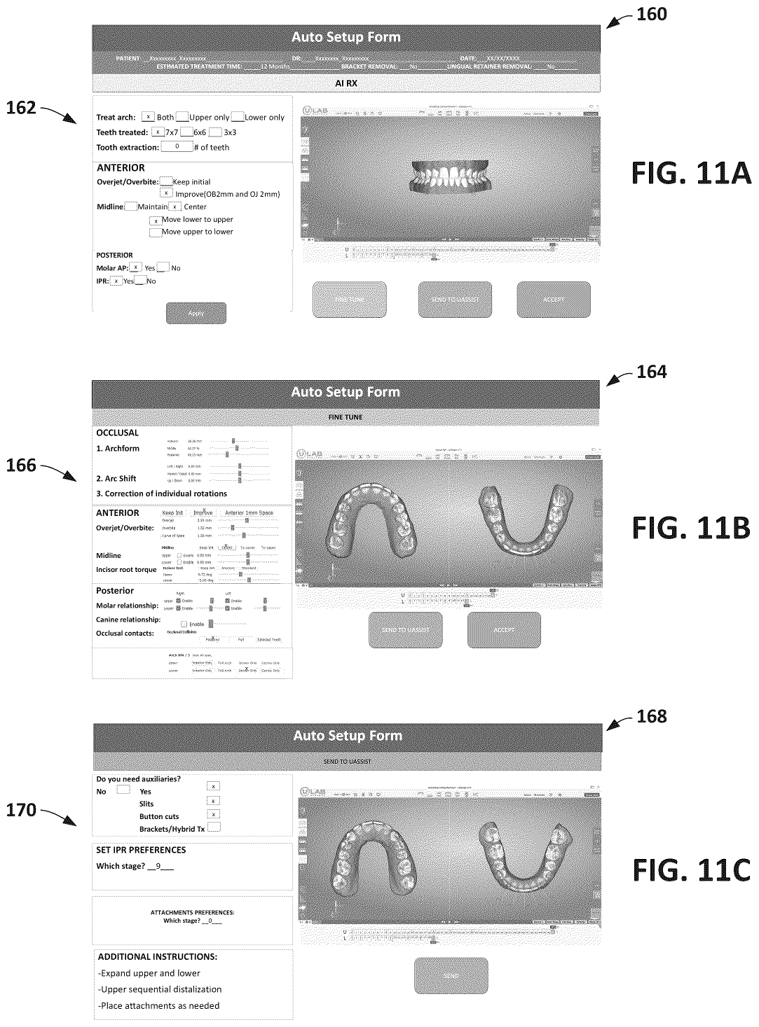

FIGS. 11A to 11C illustrate examples of the various views for each of the different options in treatment planning. FIG. 11A shows an example of a user interface 160 which may be used to facilitate the treatment planning via the automated algorithm utilizing artificial intelligence using several treatment options provided on the control interface 162. FIG. 11B shows an example of a user interface 164 which may be used to facilitate the treatment planning via the fine tuning options using several treatment options provided on the control interface 166. FIG. 11C shows an example of a user interface 168 which may be used to facilitate the treatment planning with additional auxiliary treatments using several treatment options provided on the control interface 170.

The applications of the devices and methods discussed above are not limited to the one described but may include any number of further treatment applications. Modification of the above-described assemblies and methods for carrying out the invention, combinations between different variations as practicable, and variations of aspects of the invention that are obvious to those of skill in the art are intended to be within the scope of the claims.

What is claimed is:

1. A method for optimizing a treatment plan, comprising:
receiving a digital image of a patient dentition;
automatically determining one or more movements for the treatment plan based on a state of the patient dentition in the digital image;
receiving one or more modifications relating to the treatment plan; and
accepting a finalized treatment plan.

2. The method of claim 1 wherein receiving the digital image further comprises associating the digital image to the patient dentition.

3. The method of claim 1 wherein receiving the digital image further comprises detecting and filling holes in the digital image.

4. The method of claim 3 further comprising segmenting the patient dentition in the digital image.

5. The method of claim 4 further comprising labeling the patient dentition in the digital image.

6. The method of claim 1 wherein receiving the digital image further comprises determining whether the digital image is suitable for optimization via software.

7. The method of claim 1 wherein automatically determining comprises basing the one or more movements on one or more predetermined practitioner preferences.

8. The method of claim 1 wherein receiving one or more modifications comprises automatically receiving the one or more modifications based on one or more predetermined parameters.

9. The method of claim 1 wherein accepting the finalized treatment plan further comprises preparing a prescription form.

10. The method of claim 1 further comprising ordering one or more aligners based upon the finalized treatment plan.

11. A method for optimizing a treatment plan, comprising:
receiving a digital image of a patient dentition;
automatically determining whether the digital image is suitable for optimization based upon one or more predetermined parameters;
determining one or more movements for the treatment plan based on a state of the patient dentition in the digital image;
receiving one or more modifications relating to the treatment plan; and
accepting a finalized treatment plan.

12. The method of claim 11 wherein receiving the digital image further comprises associating the digital image to the patient dentition.

13. The method of claim 11 wherein receiving the digital image further comprises detecting and filling holes in the digital image.

14. The method of claim 13 further comprising segmenting the patient dentition in the digital image.

15. The method of claim 14 further comprising labeling the patient dentition in the digital image.

16. The method of claim 11 wherein receiving the digital image further comprises determining whether the digital image is suitable for optimization via software.

17. The method of claim 11 wherein automatically determining comprises basing the one or more movements on one or more predetermined practitioner preferences.

18. The method of claim 11 wherein receiving one or more modifications comprises automatically receiving the one or more modifications based on one or more predetermined parameters.

19. The method of claim 11 wherein accepting the finalized treatment plan further comprises preparing a prescription form.

20. The method of claim 11 further comprising ordering one or more aligners based upon the finalized treatment plan.

21. The method of claim 11 further comprising ordering one or more bonding templates based upon the finalized treatment plan.
